(19) United States
(12) Patent Application Publication
(10) Pub. No.: US 2022/0195666 A1
(43) Pub. Date: Jun. 23, 2022

(54) USE OF A PLURALITY OF SALT IONIC LIQUIDS IN THE PRETREATMENT OF BIOMASS

(71) Applicants: THE REGENTS OF THE UNIVERSITY OF CALIFORNIA, Oakland, CA (US); NATIONAL TECHNOLOGY AND ENGINEERING SOLUTIONS OF SANDIA, LLC, Albuquerque, NM (US)

(72) Inventors: Hemant Choudhary, Emeryville, CA (US); Harsha D. Magurudeniya, Newport News, VA (US); John M. Gladden, Alameda, CA (US); Blake A. Simmons, San Francisco, CA (US)

(21) Appl. No.: 17/560,051

(22) Filed: Dec. 22, 2021

Related U.S. Application Data
(60) Provisional application No. 63/129,494, filed on Dec. 22, 2020.

(51) Int. Cl. D21C 3/20; C12P 19/14; D21C 3/04

(52) U.S. Cl. CPC D21C 3/20; C12P 2201/00; D21C 3/04; C12P 19/14

(57) ABSTRACT

A method to deconstruct a biomass: the method comprising: introducing a solvent comprising a plurality of salt ionic liquid (PSIL) (such as a double salt ionic liquid (DSIL)) to a biomass to dissolve at least part of solid biomass in the solvent; wherein the PSIL (or DSIL) is an organic salt comprising three or more ions, and the PSIL comprises: (i) a hard anion ionic liquid (IL) and a soft anion IL, (ii) at least one IL having a pKa value of equal to or higher than 10, or (iii) at least one IL has a low hydrogen bond donor ability.

### RELATED PATENT APPLICATIONS

This application claims priority to U.S. Provisional Patent Application Ser. No. 63/129,494, filed Dec. 22, 2020, which is hereby incorporated by reference.

### STATEMENT OF GOVERNMENTAL SUPPORT

The invention was made with government support under Contract Nos. DE-AC02-05CH11231 awarded by the U.S. Department of Energy. The government has certain rights in the invention.

### FIELD OF THE INVENTION

The present invention is in the field of biomass pretreatment.

### BACKGROUND OF THE INVENTION

Continuous efforts have been made over the last decades to transition from the use of fossil fuels as sources of chemicals and energy to renewable resources. Among these, carbon-neutral lignocellulosic biomass including human-inedible agricultural, forest, and herbaceous plant-based matter has been identified as a promising alternative for both chemicals, biomaterials, and energy (1). The main constituents of lignocellulosic biomass, namely cellulose, hemicellulose, and lignin in various ratios (depending on the biomass source), are held together by covalent and strong hydrogen bonds forming a complex matrix recalcitrant to facile depolymerization (2). The development of cutting-edge technologies to deconstruct this rigid structure into readily processable components is therefore necessary to overcome the structural complexity of the biomass and promote its efficient utilization. One of the most essential steps included in this regard is the biomass pretreatment. Several chemical pretreatment methods involving hot water, dilute acid, ionic liquid, alkali, organic solvent (organosolv), ammonia fiber expansion, among others have been explored and demonstrated (3, 4, 5).

Pretreatment with ionic liquids (ILs, salts possessing organic cations with a melting point below 100° C. (6)) is an attractive approach owing to their outstanding ability to dissolve, fractionate, and convert biopolymers (7, 8, 9). In particular, IL-based pretreatments are known to reduce cellulose crystallinity, enhance surface accessibility to (bio)catalysts, and facilitate lignin removal. Research efforts from our group and others have established that both the cation and anion in the IL governs the pretreatment mechanism involved. For instance, acetate ions ([Ace]⁻) have been observed to enhance the accessible surface area and porosity in herbaceous and woody biomass without any significant delignification (10, 11). In another study, the alkyl chain length and the aromaticity of the IL cations were found to have a profound effect on the solubility of the biopolymers in the biomass, whereas, in general, the anions affect the intra- and intermolecular interactions in these biopolymer(s) (8, 12, 13). Notably, imidazolium-based ILs have been widely investigated for biomass pretreatment and were found to be most effective on various types of biomass. However, their high cost and limited compatibility with enzymes and microorganisms commonly used in conversion processes have led the use of cholinium cation as a greener and more economical alternative (14).

Interestingly, minimal efforts have been made to integrate the respective advantageous properties of various ions in one IL to afford a clean, viable, energy intensive, and economical biomass pretreatment method. For example, the dissolution of Avicel® cellulose in a mixture of imidazolium-based ILs was recently investigated using two mechanistically similar anions, namely chloride and acetate (15). The solubility of cellulose was improved when compared to the pure ILs, probably as a result of synergy among anions with distinct characteristics.

### SUMMARY OF THE INVENTION

The present invention provides for a method to deconstruct a biomass: the method comprising: (a) introducing a solvent comprising a plurality of salt ionic liquid (PSIL) (such as a double salt ionic liquid (DSIL)) to a biomass to dissolve at least part of solid biomass in the solvent; wherein the PSIL (or DSIL) is an organic salt comprising three or more ions, and the PSIL comprises: (i) a hard anion ionic liquid (IL) and a soft anion IL, (ii) at least one IL having a pKa value of equal to or higher than 10, or (iii) at least one IL has a low hydrogen bond donor ability; (b) optionally introducing an enzyme and/or a microbe to the solubilized biomass mixture such that the enzyme and/or microbe produces a sugar from the solubilized biomass mixture; and, (c) optionally separating the sugar from the solubilized biomass mixture.

In some embodiments, the PSIL comprises four or more ions, five or more ions, or six or more ions. In some embodiments, the PSIL (or DSIL) comprises either (i) at least three ions, (ii) at least two anions and at least one cation, (iii) at least two cations and at least one anion, or (iv) at least two cations and at least two anions.

Suitable Anions Include:

carboxylate ions, such as [RCO₂]⁻, where, R can be selected from hydrogen, substituted or unsubstituted C1-20 alkyl, substituted or unsubstituted C2-20 alkenyl, substituted or unsubstituted C2-20 alkynyl, substituted or unsubstituted aryl, substituted or unsubstituted C1-20 heteroalkyl, substituted or unsubstituted C2-20 heteroalkenyl, substituted or unsubstituted C2-20 heteroalkynyl, substituted or unsubstituted heteroaryl, or substituted or unsubstituted carbonyl, substituted or unsubstituted benzyl; substituted or unsubstituted —(CH₂CH₂O)n—, wherein n is an integer from 1 to 15; or substituted or unsubstituted allyl.
[CN]⁻, [CO₃]²⁻, [HCO₃]⁻, [OH]⁻, [SH]⁻, [HSO₄]⁻, [HSO₃]⁻, [CH₃SO₄]⁻, [H₂PO₄]⁻, [HPO₄]²⁻, [PO₄]³⁻, [RPO₄]⁻, [NO₂]⁻ and halometalates including but not limited to [AlCl₄]⁻, Al₂Cl₇]⁻.

Suitable Cations Include

Ammonium cation of the structure+NR₁R₂R₃R₄, wherein R₁, R₂, R₃, and R₄ are each independently selected from hydrogen, substituted or unsubstituted C1-20 alkyl, substituted or unsubstituted C2-20 alkenyl, substituted or unsubstituted C2-20 alkynyl, substituted or unsubstituted aryl, substituted or unsubstituted C1-20 heteroalkyl, substituted or unsubstituted C2-20 heteroalkenyl, substituted or unsubstituted C2-20 heteroalkynyl, substituted or unsubstituted heteroaryl, or substituted or unsubstituted carbonyl, substituted or unsubstituted benzyl; substituted or unsubstituted —(CH₂CH₂O)n—, wherein n is an integer from 1 to 15; or substituted or unsubstituted allyl.

Phosphonium cation of the structure+PR₁R₂R₃R₄, wherein R₁, R₂, R₃, and R₄ are each independently selected from hydrogen, substituted or unsubstituted C1-20 alkyl, substituted or unsubstituted C2-20 alkenyl, substituted or unsubstituted C2-20 alkynyl, substituted or unsubstituted aryl, substituted or unsubstituted C1-20 heteroalkyl, substituted or unsubstituted C2-20 heteroalkenyl, substituted or unsubstituted C2-20 heteroalkynyl, substituted or unsubstituted heteroaryl, or substituted or unsubstituted carbonyl, substituted or unsubstituted benzyl; substituted or unsubstituted —(CH₂CH₂O)n—, wherein n is an integer from 1 to 15; or substituted or unsubstituted allyl. substituted or unsubstituted cholinium cation, substituted or unsubstituted pyridinium cation, a substituted or unsubstituted imidazolium cation, a substituted or unsubstituted morpholinium, a substituted or unsubstituted pyrrolidinium cation, a substituted or unsubstituted quinolinium cation, a substituted or unsubstituted isoquinolinium cation, or a substituted or unsubstituted morpholinium cation.

A soft anion has a large size (such as molecular weight) to charge ratio. In some embodiments, the soft anion has a size to charge ratio of equal to or more than about 50, 75, 100, 125, 150, 175, 200, 225, or 250 g/mol:1 charge. Suitable soft anions are a long chain fatty acid (such as caprylate, caprate, laurate, myristate, palmitate, stearate, arachidate, behenate, lignocerate, or cerotate), aspartate, glutamate, and lysinate. A long chain fatty acid has a main carbon chain of 8, 9, 10, 11, 12, 13, 14, 15, 16, 17, 18, 19, 20 or more carbon atoms, or any value within a range of any preceding two values. The long chain fatty acid can contain one or more C═C double bonds. The long chain fatty acid can be substituted with one, two, or more than two amine groups. In some embodiments, the soft anion has the chemical structure:

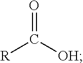

wherein R is selected from hydrogen, substituted or unsubstituted C1-20 alkyl, substituted or unsubstituted C2-20 alkenyl, substituted or unsubstituted C2-20 alkynyl, substituted or unsubstituted aryl, substituted or unsubstituted C1-20 heteroalkyl, substituted or unsubstituted C2-20 heteroalkenyl, substituted or unsubstituted C2-20 heteroalkynyl, substituted or unsubstituted heteroaryl, or substituted or unsubstituted carbonyl, substituted or unsubstituted benzyl; substituted or unsubstituted —(CH₂CH₂O)n—, wherein n is an integer from 1 to 15; or substituted or unsubstituted allyl.

Suitable soft anions include but not limited to cyanide, iodide, alkanethiolate, [CN]⁻, [CO₃]²⁻, [HCO₃]⁻, [OH]⁻, [SH]⁻, [HSO₄]⁻, [HSO₃]⁻, [CH₃SO₄]⁻, [H₂PO₄]⁻, [HPO₄]²⁻, [PO₄]³⁻, [RPO₄]⁻, [NO₂]⁻.

A hard anion has a small size (such as molecular weight) to charge ratio. In some embodiments, the hard anion has a size to charge ratio of equal to or less than about 30, 40, 50, 60, 70, 80, or 90 g/mol:1 charge. Suitable hard anions include but not limited to chlorides, nitrates, hydroxides, fluorides, chlorides, methylcarbonates, carbonates, phosphates, formate, acetate, and butyrate.

In some embodiments, suitable IL form a mixture of ions (anions and cations) in the solvent that have a pKa value of equal to or higher than 10.

In some embodiments, the solvent has a viscosity having a value equal to or less than about 0.001 cP, 0.01 cp. 0.1 cP, 1 cP, 10 cP, 20 cP, 30 cP, 40 cP, or 50 cP, or within a range of any two of the preceding values, at a temperature of about 25° C. In some embodiments, the solvent has a viscosity having a value equal to or less than about 0.001 cP, 0.01 cp, 0.1 cP, 1 cP, 10, cP, 50 cP, 100 cP, 150 cP, 200 cP, 250 cP, 300 cP, 350 cP, 400 cP, 450 cP, 500 cP, 550 cP, or 600 cP, or within a range of any two of the preceding values, at a temperature of about 90° C. In some embodiments, the solvent has a viscosity having a value equal to or less than about 40 cP, 45 ep, 50 cP, 55 cP, or 60 cP at a temperature of about 90° C.

In some embodiments, the solvent has a boiling point having a value equal to or less than about 40° C., 50° C., 60° C., 70° C. 80° C., 90° C. 100° C., 110° C., 120° C., 130° C., 140° C., 150° C., 160° C., 170° C., 180° C., 190° C., or 200° C., or within a range of any two of the preceding values.

In some embodiments, the solvent has a viscosity having a value equal to or less than about 50 cP at a temperature of about 90° C.

In some embodiments, the solvent has a viscosity having a value equal to or less than about 600 cP at a temperature of about 25° C.

In some embodiments, the one or more individual components are selected from the group consisting of molecules that can form ILs: cations (such as an amine containing molecules such as ethanolamine, choline, and the like) and anions (such as mineral and organic acids, such as sulfuric acid, acetic acid, and the like). In some embodiments, the introducing step (a) comprises introducing two or individual components to the biomass, wherein the two or individual components form an IL, or mixture thereof. In some embodiments, the components already present in the biomass are components that are naturally found in a biomass.

In some embodiments, the introducing step (a) comprises introducing each individual component separately to the biomass.

In some embodiments, the method further comprises ensiling a biomass, prior to the introducing step (a), to produce an ensiled biomass comprising one or more organic acids, wherein the ensile biomass is the biomass of the introducing step (a). In some embodiments, the ensiled biomass comprises equal to or more than about 10%, 20%, 30%, or 40% by weight of the one or more organic acids. In some embodiments, the one or more organic acids comprises an alkanoic acid. In some embodiments, the alkanoic acid is lactic acid, acetic acid, butyric acid, or propionic cid, or a mixture thereof.

In some embodiments, the method further comprises (b) introducing an enzyme and/or a microbe to the solubilized biomass mixture such that the enzyme and/or microbe produces a sugar from the solubilized biomass mixture.

In some embodiments, the method further comprises (c) separating the sugar from the solubilized biomass mixture.

In some embodiments, the method results in a yield of equal to or more than about 70%, 75%, 80%, 85%, 90%, or 95% of sugar from the biomass. In some embodiments, the sugar is a hexose or a pentose. In some embodiments, the hexose is a glucose. In some embodiments, the pentose is a xylose.

In some embodiments, step (a) does not comprise, or lacks, introducing or adding any water to the biomass or mixture. In some embodiments, the amount of water in the mixture, excluding or including water or moisture naturally found in the biomass is no more than about 10%, 9%, 8%, 7%, 6%, 5%, 4%, 3%, 2%, 1%, 0.5%, or 0.1% by weight or volume of the mixture.

In some embodiments, the PSIL comprises an ionic liquid (IL) having the formula [aC₁+bC₂+ . . . +zCₙ][αA₁+βA₂+ . . . +ωAₙ₋₁+(1−α−β− . . . −ω)Aₙ]; wherein C₁, C₂, . . . and Cₙ are organic cations and at least one organic cation is an alkylammonium, an arylammonium, an allylammonium, an imidazolium, a pyridinium, a phosphonium, a sulphonium, or a combination thereof; A₁, A₂, . . . Aₙ are anions, wherein at least one of the anions is a hard anion comprising a carboxylic acid or an amino acid; a, b, . . . and z are independently a number from about 0 to 20; and a sum of a, b, . . . and z is greater than 0; a sum of α+β+ . . . +ω is a number greater than zero, such as from about 0.01 to 0.99.

In some embodiments, the PSIL comprises an ionic liquid (IL) having the formula [mC₁+nC₂][xA₁+(1−x)A₂)]; wherein C₁ and C₂ are organic cations and at least one organic cation is an alkylammonium, an arylammonium, an allylammonium, an imidazolium, a pyridinium, a phosphonium, a sulphonium, or a combination thereof; A₁ and A₂ are anions, wherein at least one of the anion is a hard anion comprising a carboxylic acid or an amino acid; m and n are independently a number from about 0 to 5; and a sum of m and n is greater than 0; x is a number from about 0.01 to 0.99. In some embodiments, m and n are independently about 0, 1, 2, 3, 4, or 5. In some embodiments, x is about 0.01, 0.05, 0.1, 0.2, 0.3, 0.4, 0.5, 0.6, 0.7, 0.8, 0.9, or 0.99, or any number within a range of any two preceding numbers.

In some embodiments, the carboxylic acid is an acetate, propionate, butyrate, valerate, caproate, enanthate, caprylate, pelargonate, or caprate, or a mixture thereof. In some embodiments, the amino acid is a naturally occurring amino acid, such as lysine or glycine. In some embodiments, the IL is a liquid at a temperature from about −80° C. to about 150° C. In some embodiments, the combination of hard anion renders the hydrogen bond basicity of the IL at equal to or more than about 0.25 at about 90° C. In some embodiments, equal to or more than 50% of the lignin in the solubilized biomass mixture has a molecular weight within a range of 1000 to 15000 Da.

In some embodiments, the solubilized biomass mixture has fewer guaiacol and/or derivatives thereof, such as compared to a pretreatment using only one salt ionic liquid. An example is the solubilized biomass mixture produced by Treatment 1, 2, or 3 described in Example 2 herein. As guaiacol and guaiacol derivatives are odoriferous, a solubilized biomass mixture that has fewer or no guaiacol and guaiacol derivatives are relatively less odoriferous.

One of the major issues in lignin upgradation after biomass fractionation (during the pretreatment step) is lignin condensation reaction that is the formation of new intermolecular C—C bonds between lignin fragments. This results in a decrease in readily cleavable aryl-ether linkages in the lignin structure. In order to preserve native-like lignin structure, the use of capping agents are sometimes strategically introduced to stabilize the intermediates or reactive sites of lignin fragments. Commonly used capping agents include, but are limited to, (a) formaldehyde (or other aldehydes) to form cyclic acetals, and (b) boric acid or dimethyl sulfate for selective masking of aryl hydroxyl groups. In some embodiments, the method does not comprise, or lacks, introducing or adding a capping agent to the biomass, solvent, and/or solubilized biomass mixture. Treatment 3 produces a profile of lignin that is substantially similar to the profile of native or native-like lignin, such as the profile of the distribution of molecular weight of the lignin (such as shown in FIG. 17). For example, the profile of lignin that has a peak similar to that for untreated or pine biomass for the molecule:

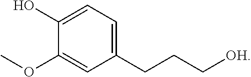

In some embodiments, a similar peak is a peak that has value at least 80%, 85%, 90%, or 95% of the reference peak, such as that for untreated or pine biomass.

The present invention provides for compositions and methods described herein. In some embodiments, the compositions and methods further comprise steps, features, and/or elements described in U.S. patent application Ser. No. 16/737,724, hereby incorporated by reference in its entirety.

In some embodiments, the method is a one-pot method, and does not require any solid-liquid separation step. In some embodiments, the one-pot method does not require adjustment of the pH level in the one-pot composition. In some embodiments, the one-pot method does not require any dilution, or addition of water or medium, after pretreatment and/or before saccharification and fermentation. In some embodiments, the reaction of the enzyme and the growth of the microbe occur in the same one-pot composition. In some embodiments, the IL is renewable as it can be continuous in use. In some embodiments, the one-pot method can produce a yield of sugar that is equal to or more than about 50%, 60%, 70%, 75%, or 80%, or any other value described herein.

In some embodiments, using bio-compatible solvents enables a one-pot biomass conversion which eliminates the needs of mass transfer between reactors and the separation of solid and liquid. In some embodiments, the method does not require recycling any catalyst and/or enzyme. In some embodiments, the method requires less water usage than current biomass pretreatment. The method can produce fuels/chemicals at a higher titer and/or yield in a single vessel without any need for intermediate units of mass transfer and/or solid/liquid separation.

The present invention provides for compositions and methods described herein.

In some embodiments, the compositions and methods further comprise steps, features, and/or elements described in U.S. patent application Ser. No. 16/737,724, hereby incorporated by reference in its entirety.

The present invention provides for a method to enhance biomass pretreatment efficacy using a plurality of salt ionic liquids (PSIL), such as double salt ionic liquids (DSIL). A plurality of salt ionic liquids, such as DSIL, comprises organic salts comprising three or more ions. In some embodiments, when imidazolium, cholinium, carboxylate (such as acetate), and/or lysinate ions are combined, efficient pretreatment of softwood is achieved releasing at least about 80% glucose and/or at least about 70% xylose at at least about 20 wt % solid loading. In some embodiments, the PSIL or DSIL comprises palmitate and/or octanoate anions.

In some embodiments, the PSIL (or DSIL) comprises a 1-alkyl-3-alkylimidazolium alkanoate, such as 1-ethyl-3-methylimidazolium acetate ([C2mim][OAc]), combined with a cholinium amino acid, such as cholinium lysinate ([Chol][Lys]). In some embodiments, the DSIL comprises 1-ethyl-3-methylimidazolium acetate ([C2mim][OAc]) combined with cholinium lysinate ([Chol][Lys]). In some embodiments, there is solid loading of the biomass at about 5%, 10%, 15%, 20%, 25%, or 30%, or within a range of value between any two of the preceding values.

At 20% solid loading, the pretreatment efficacy of [Chol][Lys] is improved from 49% and 43% to about 80% and about 70% of glucose and xylose, respectively, by doping it with an amount of ([C2mim][OAc]). In another embodiment, the introduction of palmitate as a secondary anion into cholinium lysinate afforded [Chol][Lys][Pal] DSIL improved the pretreatment efficacy and reduced the microbial toxicity of [Chol][Lys].

The present invention provides for using different anions (and/or cations) with specific function such as stronger basicity or higher lignin solubility in one formulation of the PSIL (or DSIL) to achieve more economical and sustainable biomass pretreatment methodologies.

The present invention is useful for converting waste biomass (for example, from agricultural residues, wood/paper/pulping, grasses, and the like) into biofuels and/or bioproducts. The method is useful in achieving higher concentrations of fermentable sugar(s) while leaving the residual lignin for the production of valuable chemicals.

The present invention has one or more of the following advantages: (1) Ionic liquids developed inexpensive reagents. (2) Highly compatibility with downstream processes. (3) Reduced amount of ILs required for effective pretreatment. (4) Biomass type versatility. (5) Recycling of reagents.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. (A) A method for producing dry solid from a biomass, such as pine. (B) Glucose (black) and xylose (gray) yields after enzymatic hydrolysis of untreated or pretreated pine with 1-ethyl-3-methylimidazolium acetate (EA), cholinium lysinate (CL) and 1:1 (w/w) mixture of EA and CL. Pretreatment conditions: pine (20 wt %), IL (80 wt %), 140° C., 3 h. Saccharification conditions: pine (5 wt %), 10 mg enzyme (CTec3:HTec3, 9:1 v/v) per g sorghum, 50° C., 72 h.

FIG. 2. COSMO-RS predicted logarithmic activity coefficients (ln(γ)) of lignin in various cholinium-based ILs/DSILs.

FIG. 3. Left Y-axis. Glucose (black bars) and xylose (gray bars) yields after enzymatic hydrolysis of untreated or IL/DSIL pretreated sorghum. Right Y-axis. Lignin removal efficiency (o) after pretreatment with cholinium-based IL/DSIL. Pretreatment conditions: sorghum (20 wt %), IL/DSIL (80 wt %), 140° C., 3 h. Saccharification conditions: sorghum (5 wt %), 10 mg enzyme (CTec3:HTec3, 9:1 v/v) per g sorghum, 50° C., 72 h.

FIG. 4. (A) Bisabolene titers after a 4-day incubation, and (B) growth curves of Rhodosporidium toruloides during a 2-day incubation in hydrolysate obtained using [Lys][Pal](black) and [Lys] (gray) as pretreatment solvents.

FIG. 5. Anions used to prepare cholinium-based DSILs evaluated in this study.

FIG. 6. Glucan (black), xylan (gray), and lignin (dark gray) content of untreated and pretreated pine with 1-ethyl-3-methylimidazolium acetate (EA), cholinium lysinate (CL) and 1:1 (w/w) mixture of EA and CL. Pretreatment conditions: 2 mm pine (20 wt %), IL (80 wt %), 140° C., 3 h.

FIG. 7. Glucan (black), xylan (gray), and lignin (dark gray) content of untreated and pretreated sorghum with cholinium-based IL/DSIL. Pretreatment conditions: 2 mm Sorghum (20 wt %), IL/DSIL (80 wt %), 140° C., 3 h.

FIG. 8. Experimental and COSMO-RS-based developed models predicted lignin (grass) solubility in cholinium-based ILs and DSILs. (A) Model developed considering activity coefficient; Lignin solubility=a₀+a₁*(γ) and (B) Model developed considering activity coefficient, excess enthalpy, and hydrogen bonding energy; Lignin solubility=b₀+(b₁/exp(H^{E}))+(b₂/exp(γ))+(b₃*(HB_energy)).

FIG. 9. Adopted structure of lignin including all the major linkages as present in the native lignin in grass.

FIG. 10. FT-IR of cholinium-based ILs and precursors. (A) Cholinium Lysinate ([Ch][Lys]), (B) Cholinium Acetate ([Ch][Ace]), (C) Cholinium Octanoate ([Ch][Oct]), and (D) Cholinium Palmitate ([Ch][Pal]).

FIG. 11. FT-IR of lysinate-containing IL/DSILs. Top to bottom: lysine, [Ch][Lys], [Ch][Lys][Ace], [Ch][Lys][Oct], and [Ch][Lys][Pal].

FIG. 12. FT-IR of acetate-containing IL/DSILs. Top to bottom: acetic acid, [Ch][Ace], [Ch][Lys][Ace], [Ch][Ace][Oct], and [Ch][Ace][Pal].

FIG. 13. FT-IR of octanoate-containing IL/DSILs. Top to bottom: octanoic acid, [Ch][Oct], [Ch][Lys][Oct], [Ch][Ace][Oct], and [Ch][Oct][Pal].

FIG. 14. FT-IR of palmitate-containing IL/DSILs. Top to bottom: palmitic acid, [Ch][Pal], [Ch][Lys][Pal], [Ch][Ace][Pal], and [Ch][Oct][Pal].

FIG. 15. Various lignin obtained from Pine biomass after IL/DSIL pretreatment and enzymatic hydrolysis (EH).

FIG. 16. Powder X-ray diffraction patterns of lignin obtained after various IL/DSIL treatment demonstrating decrease in the cellulosic content and change in crystalline phases. EH is enzymatic hydrolysis.

FIG. 17. Pyro-GC analysis of the lignin obtained after various treatments showing the retention of units and linkages after treatment 3 as in the native pine. The sugar component in the pine is reduced after enzymatic hydrolysis (EH).

FIG. 18. Thermogravimetric analysis of lignin obtained after various treatments demonstrating distinct thermal profile.

FIG. 19. Pd/ZrP catalysis on pine. (A) The results from different pretreatment conditions. (B) The distribution of molecules by molecular weight after pretreatment. The reaction conditions used are: IL-processed-pine lignin (0.35 g), Pd/ZrP (0.1 g), iPrOH:MeOH (2:1 v/v, 10 mL), 300° C., N₂ (18 bar), 500 rpm.

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In some embodiments, the introducing step (a) comprises contacting a biomass and one or more individual components of the solvent sequentially, or all or part in step(s). In some embodiments, the contacting step comprises introducing, adding and/or mixing the biomass with the one or more individual components of the solvent, or vice versa.

In some embodiments, the introducing one or more individual components of the solvent to a biomass takes place in a vessel and homogenized. In some embodiments, the loading is solid loading and controlled at about 5%, 10%, 15%, 20%, 25%, 30%, 35%, or 40%, or a range within any two preceding values. In some embodiments, the biomass and IL and/or solvent are heated, such as to 100° C., 110° C., 120° C., 130° C., 140° C., 150° C., 160° C., 170° C., 180° C., 190° C., 200° C., 200° C., 212° C., or a range within any two preceding values, for a period of time, such as about 1 h, 2 h, 3 h, 4 h, or 5 h, or a range within any two preceding values. In some embodiments, after pretreatment, the mixture is cooled, such as for a period of about at least 30 mins, such as at room temperature, or about 25° C., and/or then washed at least about 1 X, 2X, 3 X, 4 X, or 5 X with water, such as deionized water. In some embodiments, the resulting solid is recovered, such as separating the solid portion with the liquid portion.

In some embodiments, the biomass is a lignocellulosic biomass. In some embodiments, the vessel is made of a material that is inert, such as stainless steel or glass, that does not react or interfere with the reactions in the pretreatment mixture.

In some embodiments, the method uses a one-pot methodology, for example, using method steps and compositions as taught in U.S. patent application Ser. No. 16/737,724 (which is incorporated by reference). In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the method produces a sugar and a lignin from the biomass. The lignin can further be processed to produce a non-naturally occurring compound. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps (a) and (b), and optionally steps (c) and/or (d), are continuous, the one-pot composition is in a steady state.

### Ionic Liquid

Ionic liquids (ILs) are salts that are liquids rather than crystals at room temperatures. It will be readily apparent to those of skill that numerous ILs can be used in the present invention. In some embodiments of the invention, the IL is suitable for pretreatment of the biomass and for the hydrolysis of cellulose by thermostable cellulase. Suitable ILs are taught in ChemFiles (2006) 6(9) (which are commercially available from Sigma-Aldrich, Milwaukee, Wis.). Such suitable ILs include, but are not limited to, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanate” is an alkanate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanate” is an alkanate comprising from 1 to 4 carbon atoms. In some embodiments, the “alkanate” is an acetate. In some embodiments, the halide is chloride.

In some embodiments, the IL includes, but is not limited to, 1-ethyl-3-methylimidazolium acetate (EMIN Acetate), 1-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl₄), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like.

In some embodiments, the ionic liquid is a chloride ionic liquid. In other embodiments, the ionic liquid is an imidazolium salt. In still other embodiments, the ionic liquid is a 1-alkyl-3-imidazolium chloride, such as 1-ethyl-3-methylimidazolium chloride or 1-butyl-3-methylimidazolium chloride.

In some embodiments, the ionic liquids used in the invention are pyridinium salts, pyridazinium salts, pyrimidium salts, pyrazinium salts, imidazolium salts, pyrazolium salts, oxazolium salts, 1,2,3-triazolium salts, 1,2,4-triazolium salts, thiazolium salts, isoquinolium salts, quinolinium salts isoquinolinium salts, piperidinium salts and pyrrolidinium salts. Exemplary anions of the ionic liquid include, but are not limited to halogens (e.g., chloride, floride, bromide and iodide), pseudohalogens (e.g., azide and isocyanate), alkyl carboxylate, sulfonate, acetate and alkyl phosphate.

Additional ILs suitable for use in the present invention are described in U.S. Pat. Nos. 6,177,575; 9,765,044; and, 10,155,735; U.S. Patent Application Publication Nos. 2004/0097755 and 2010/0196967; and, PCT International Patent Application Nos. PCT/US2015/058472, PCT/US2016/063694, PCT/US2017/067737, and PCT/US2017/036438 (all of which are incorporated in their entireties by reference). It will be appreciated by those of skill in the art that others ILs that will be useful in the process of the present invention are currently being developed or will be developed in the future, and the present invention contemplates their future use. The ionic liquid can comprise one or a mixture of the compounds.

In some embodiments, the IL is a protic ionic liquid (PIL). Suitable protic ionic liquids (PILs) include fused salts with a melting point less than 100° C. with salts that have higher melting points referred to as molten salts. Suitable PPILs are disclosed in Greaves et al. “Protic Ionic Liquids: Properties and Applications” Chem. Rev. 108(1):206-237 (2008). PILs can be prepared by the neutralization reaction of certain Brønsted acids and Brønsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7, C8, C9, or C10 chain. In some embodiments, the carboxylate is substituted with one or more hydroxyl groups. In some embodiments, the PIL is a hydroxyethylammonium acetate.

In some embodiments, the protic ionic liquid (PIL) is disclosed by U.S. Patent Application Publication No. 2004/0097755, hereby incorporated by reference.

Suitable salts for the method include combinations of organic ammonium-based cations (such as ammonium, hydroxyalkylammonium, or dimethylalkylammonium) with organic carboxylic acid-based anions (such as acetic acid derivatives (C1-C8), lactic acid, glycolic acid, and DESs such as ammonium acetate/lactic acid).

Suitable IL, such as distillable IL, are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “CO₂-based Alkyl Carbamate Ionic Liquids as Distillable Extraction Solvents”, ACS Sustainable Chem. Engin. 2:31724-1728 (2014), all of which are hereby incorporated by reference.

Suitable PIL, such as distillable PIL, are disclosed in Idris et al. “Distillable Protic Ionic Liquids for Keratin Dissolution and Recovery”, ACS Sustainable Chem. Engin. 2:1888-1894 (2014) and Sun et al. “One-pot integrated biofuel production using low-cost biocompatible protic ionic liquids”, Green Chem. 19(13):3152-3163 (2017), all of which are hereby incorporated by reference.

In some embodiments, the PILs are formed with the combination of organic ammonium-based cations and organic carboxylic acid-based anions. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application. In some embodiments, the PIL (such as hydroxyethylammonium acetate—[Eth][OAc]) is an effective solvent for biomass pretreatment and is also relatively cheap due to its ease of synthesis (Sun et al., Green Chem. 19(13):3152-3163 (2017)).

In some embodiments, the solubilizing is full, near full (such as at least about 70, 80, or 90%), or partial (such as at least about 10, 20, 30, 40, 50, or 60%). In some embodiments, the one-pot composition is a slurry. When the steps described herein are continuous, the one-pot composition is in a steady state.

In some embodiments, the introducing step comprises heating the mixture comprises increasing the temperature of the solution to a value within a range of about 75° C. to about 125° C. In some embodiments, the heating step comprises increasing the temperature of the solution to a value within a range of about 80° C. to about 120° C. In some embodiments, the heating step comprises increasing the temperature of the solution to a value within a range of about 90° C. to about 110° C. In some embodiments, the heating step comprises increasing the temperature of the solution to about 100° C.

### Enzyme

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteiii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### Biofuel

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

### Biomass

The biomass can be obtained from one or more feedstock, such as softwood feedstock, hardwood feedstock, grass feedstock, and/or agricultural feedstock, or a mixture thereof. In some embodiments, the biomass is a lignocellulosic biomass comprising cellulose, hemicellulose, and lignin in various ratios (depending on the biomass source). The cellulose, hemicellulose, and lignin are held together by covalent and strong hydrogen bonds forming a complex matrix recalcitrant to facile depolymerization.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picea rubens, Picea sitchensis, Picea glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hombeam; Hophornbeam; Ipê; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandi; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoumd; Oregon Myrtle; California Bay Laurel; Pear; Poplar (e.g. P. balsamifera, P. nigra, Hybrid Poplar (Populus x canadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C₄ or C₃ grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, sorghum, rice, soybeans, hay, potatoes, cotton, or sugarcane. The feedstock can arise from the harvesting of crops from the following practices: intercropping, mixed intercropping, row cropping, relay cropping, and the like.

In some embodiments, the biomass is an ensiled biomass. In some embodiment, the biomass is ensiled by placing the biomass in an enclosed container or room, such as a silo, or by piling it in a heap covered by an airproof layer, such as a plastic film. The biomass undergoing the ensiling, known as the silage, goes through a bacterial fermentation process resulting in production of volatile fatty acids. In some embodiment, the ensiling comprises adding ensiling agents such as sugars, lactic acid or inculants. In some embodiments, the ensiled biomass comprises one or more toxic compounds. In some embodiments, when ensiled biomass comprises one or more toxic compounds, the microbe is resistant to the one or more toxic compounds.

### REFERENCES CITED HEREIN

1. Biorefinery: Integrated sustainable processes for biomass conversion to biomaterials, biofuels, and fertilizers, (Eds.: J. -R. Bastidas-Oyanedel, J. E. Schmidt), Springer Nature, Cham, 2019.
2. L. J. Gibson, J. R. Soc. Interface 2012, 9, 2749.
3. S. H. Mood, A. H. Golfeshan, M. Tabatabaei, G. S. Jouzani, G. H. Najafi, M. Gholami, M. Ardjmand, Renewable Sustainable Energy Rev. 2013, 27, 77.
4. Z. Zhou, F. Lei, P. Li, J. Jiang, Biotechnol. Bioeng. 2018, 115, 2683.
5. B. Kumar, N. Bhardwaj, K. Agarwal, V. Chaturvedi, P. Verma, Fuel Process. Technol. 2020, 199, 106244.
6. J. D. Holbrey and R. D. Rogers in Ionic Liquids in Synthesis, 1st edition (Eds.: P. Wasserscheid, T. Welton), Wiley-VCH Verlag GmbH & Co. KGaA, Weinheim, 2002, pp. 41-55.
7. Z. Usmani, M. Sharma, P. Gupta, Y. Karpichev, N. Gathergood, R. Bhatt, V. K. Gupta, Bioresour. Technol. 2020, 304, 123003.
8. C. G. Yoo, Y. Pu, A. J. Ragauskas, Curr. Opin. Green Sustain. Chem. 2017, 5, 5.
9. D. J. G. P. van Osch, L. J. B. M. Kollau, A. van den Bruinhorst, S. Asikainen, M. A. A. Rocha, M. C. Kroon, Phys. Chem. Chem. Phys. 2017, 19, 2636.
10. K. M. Torr, K. T. Love, B. A. Simmons, S. A. Hill, Biotechnol. Bioeng. 2016, 113, 540.
11. T. Raj, R. Gaur, B. Y. Lamba, N. Singh, R. P. Gupta, R. Kumar, S. K. Puri, S. S. V. Ramakumar, Bioresour. Technol. 2018, 249, 139.
12. J. Stanton, Y. Xue, P. Pandher, L. Malek, T. Brown, X. Hu, D. Salas-de la Cruz, Int. J. Biol. Macromol. 2018, 108, 333.
13. K. Yavir, L. Marcinkowski, R. Marcinkowska, J. Namiesnik, A. Kloskowski, Anal. Chim. Acta 2019, 1054, 1.
14. X. -D. Hou, T. J. Smith, N. Li, M. -H. Zong, Biotechnol. Bioeng. 2012, 109, 2484.
15. I. A. Masiutin, A. V. Golyshkin, A. A. Litvin, A. A. Novikov, M. A. Kotelev, E. V. Ivanov, V. A. Vinokurov, Cell. Chem. Technol. 2018, 52, 51.
16. N. Sun, R. Parthasarathi, A. M. Socha, J. Shi, S. Zhang, V. Stavila, K. L. Sale, B. A. Simmons, S. Singh, Green Chem. 2014, 16, 2546.
17. I. Chiarotto, L. Mattiello, F. Pandolfi, D. Rocco, M. Feroci, Front. Chem. 2018, 6, 355 and references therein.
18. G. Chatel, J. F. B. Pereira, V. Debbeti, H. Wang, R. D. Rogers, Green Chem. 2014, 16, 2051.
19. H. Choudhary, J. Pernak, J. L. Shamshina, M. Niemczak, R. Giszter, L. Chrzanowski, T. Praczyk, K. Marcinkowska, O. A. Cojocaru, R. D. Rogers, ACS Sustainable Chem. Eng. 2017, 5, 6261.
20. Y. -R. Liu, K. Thomsen, Y. Nie, S. -J. Zhang, A. S. Meyer, Green Chem. 2016, 18, 6246.
21. A. Casas, J. Palomar, M. V. Alonso, M. Oliet, S. Omar, F. Rodriguez, Ind. Crops Prod. 2012, 37, 155.
22. I. Gelfand, S. K. Hamilton, A. N. Kravchenko, R. D. Jackson, K. D. Thelen, G. P. Robertson, Environ. Sci. Technol. 2020, 54, 2961.
23. J. -M. Andanson, E. Bordes, J. Devémy, F. Leroux, A. A. H. Pádua, M. F. C. Gomes, Green Chem. 2014, 16, 2528.
24. Also see FIG. 8.
25. M. Mohan, P. Viswanath, T. Banerjee, V. V. Goud, Mol. Phys. 2018, 116, 2108.
26. F. Xu, J. Sun, N. V. S. N. M. Konda, J. Shi, T. Dutta, C. D. Scown, B. A. Simmons, S. Singh, Energy Environ. Sci. 2016, 9, 1042.
27. J. Sun, N. V. S. N. M. Konda, R. Parthasarathi, T. Dutta, M. Valiev, F. Xu, B. A. Simmons, S. Singh, Green Chem. 2017, 19, 3152.

### Example 1

Multiple Ions in an Ionic Liquid Improve the Biomass Pretreatment Efficacy

Eccentric ionic liquid (IL) systems comprising of multiple ions known to possess distinctive pretreatment mechanisms were developed and evaluated for woody and grassy biomass. Molecular simulations and experimental results established the synergistic advantages of combining individual components in these systems. For pine (woody) biomass pretreatment with IL, the combination of imidazolium, cholinium, acetate, and lysinate ions achieved 80% glucose and 70% xylose yields at high biomass loading. In another context using sorghum biomass, an IL system comprising of cholinium, lysinate, and palmitate ions not only released ˜98% glucose yield but was also found to be biocompatible in a one-pot configuration producing the biofuel precursor bisabolene, using an engineered strain of the yeast Rhodosporidium toruloides.

Herein, we employed 1-ethyl-3-methyl-imidazolium acetate ([C₂mim][Ace]) and cholinium lysinate ([Ch][Lys]) in combination for the pretreatment of pine (Pinus radiata), a challenging softwood biomass. 20 wt % pine was suspended in 1:1 (w/w) mixture of [C₂mim][Ace] and [Ch][Lys] to afford a total IL loading of 80 wt %. The pine-IL slurry was then heated at 140° C. for 3 h. Pine was also pretreated with pure ILs as a control. Effective pretreatment of biomass with most ILs have been reported at temperatures between 120 and 160° C. (7, 8), and thereby we chose 140° C. for our experiments. The pretreated biomass was washed extensively with water to obtain IL-free pretreated pine solids. After washing, yields of solids corresponding to 81.4%, 77.1% and 77.2% were recovered from pretreatments using [C₂mim][Ace], [Ch][Lys], and 1:1 mixture of ILs, respectively (see FIG. 6).

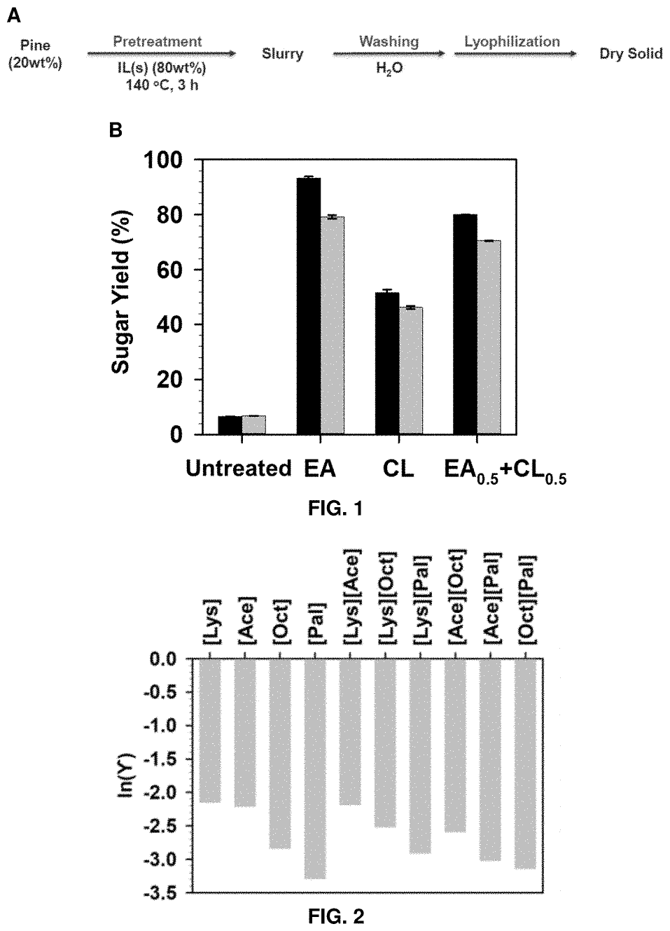

The pretreatment efficacy was measured in terms of holocellulose digestibility using commercial enzyme cocktails (Novozymes Cellic® CTec3 and HTec3) and plotted as FIG. 1. The enzymatic hydrolysis was carried out at a protein loading of 10 mg per g of biomass at 50° C. for 72 h. All IL pretreatments resulted in significantly faster saccharification rates compared to the untreated pine (6.6% glucose and 6.8% xylose). Consistent with previous reports, [C₂mim][Ace] could effectively pretreat softwood yielding 93.2% glucose and 79.2% xylose; possibly by enhancing the accessible surface area (10). [Ch][Lys], on the other hand, released only 51.6% and 46.3% glucose and xylose, respectively. These values are considerably lower than those typically obtained when using this IL with grassy biomass, demonstrating the importance of developing new approaches to deconstruct feedstocks such as pine (16). Interestingly, 80.1% glucose and 70.5% xylose was noted with a 1:1 w/w mixture of [C₂mim][Ace] and [Ch][Lys] under identical conditions. It must be highlighted that strong molecular bases are known to deprotonate acidic proton of imidazolium ILs forming carbenes and adducts (17), which consequently render such ILs ineffective for pretreatment. This was not observed in our case when [C₂mim][Ace] was used in combination with a stronger IL base, [Ch][Lys].

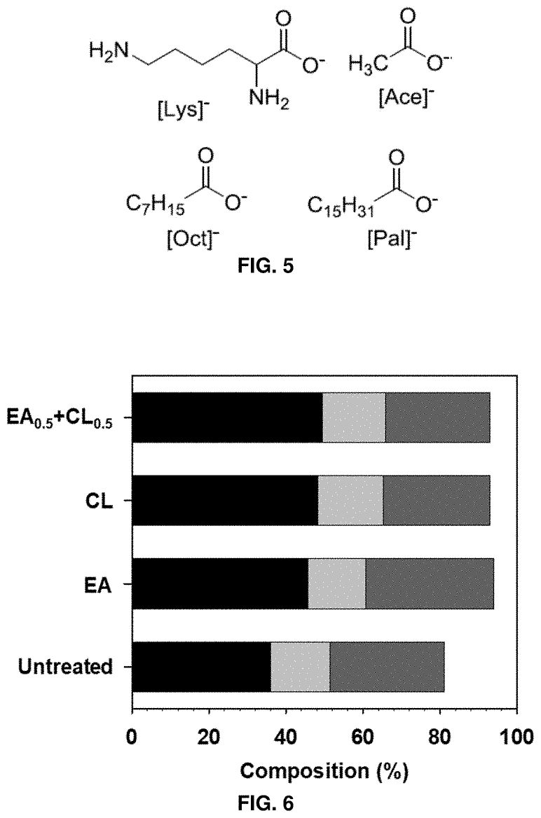

The incorporation of multiple ions with known distinct pretreatment mechanisms in an IL paves the path to develop new strategies to boost the pretreatment efficiency while reducing the cost associated with the pretreatment step. In order to further explore the concept, here we use a unique tool called “double salt ionic liquids” (DSILs; systems containing three or more ions often possessing unexpected physicochemical properties), developed by the IL community (18, 19). In this study, we have synthesized the cholinium-based DSIL employing lysinate, acetate, octanoate ([Oct]⁻), and palmitate ([Pal]⁻) anions (FIG. 5), since the anions have been known to play a predominant role in biomass pretreatment (8, 10). Lysinate has been observed to selectively dissolve lignin during biomass pretreatment, while acetate is a stronger base known to effectively cleave intermolecular H-bonding (16). We anticipate palmitate (C₁₆ acid-derived anion) to have higher lignin interactions due to the hydrophobicity of the longer alkyl chain. Also, the hydrophobicity that is being imparted to the IL might facilitate its recycling. Octanoate, a C₈ acid-derived anion is believed to possess properties unique to both acetate and palmitate.

Cholinium-based ILs and DSILs were synthesized by acid-base reactions of cholinium hydroxide in methanol and appropriate acid or mixture of acids (see Supporting Information). [Ch][Lys], cholinium acetate ([Ch][Ace]), cholinium octanoate ([Ch][Oct]), and cholinium palmitate ([Ch][Pal]) were obtained by treating one equivalent of cholinium hydroxide with one equivalent of lysine, acetic, octanoic, and palmitic acids, respectively. For DSIL synthesis, cholinium hydroxide was reacted with a 1:1 mixture of two acids (added at once) with the total molar amount of the acids being equal to that of the hydroxide to yield DSILs with a general formula, [Ch][A1]_{0.5}[A2]_{0.5}, where A1 and A2 are the anions from two different acids. For the sake of simplicity, these will be represented as [Ch][[A1][A2], hereafter. DSILs synthesized were cholinium lysinate acetate ([Ch][Lys][Ace]), cholinium lysinate octanoate ([Ch][Lys][Oct]), cholinium lysinate palmitate ([Ch][Lys][Pal]), cholinium acetate octanoate ([Ch][Ace][Oct]), cholinium acetate palmitate ([Ch][Ace][Pal]), and cholinium octanoate palmitate ([Ch][Oct][Pal]). The identity and purity of the synthesized ILs and DSILs was established by NMR and thermal analysis (see Supporting Information).

COnductor like Screening MOdel for Real Solvent (COSMO-RS) calculations has been embraced on several occasions to explore the viability of a new solvent candidate in biomass pretreatment. Most of the previous studies have concluded that logarithmic activity coefficient (ln(γ)) is a dominant parameter in predicting dissolution properties of the solute, while others have also considered the excess enthalpy (H^{E}) along with ln(γ) (20, 21). Herein, we predicted the ln(γ) of lignin in various cholinium-based IL/DSIL to test the hypothesis through studying the intra- and intermolecular interactions between lignin and IL/DSIL (FIG. 2). Typically, lower logarithmic activity coefficients (ln(γ)) implies stronger interactions (i.e., higher dissolution) of the solute with in the solvent. Based on these predictions, palmitate containing ILs/DSILs were pronounced better pretreatment solvents as far as lignin dissolution was concerned.

A very recent study pointed out that the use of grass as a feedstock over woody biomass could potentially assuage the effects of global warming (22). Considering this fact, the pretreatment effectiveness of prepared IL/DSIL was evaluated on sorghum (Sorghum bicolor; grass) rather than pine. Similar to aforementioned, 20 wt % sorghum was mixed with IL (or DSIL) and heated at 140° C. for 3 h. The slurry, thus obtained, was washed with water-ethanol (1:1 v/v) to remove IL/DSIL from the biomass.

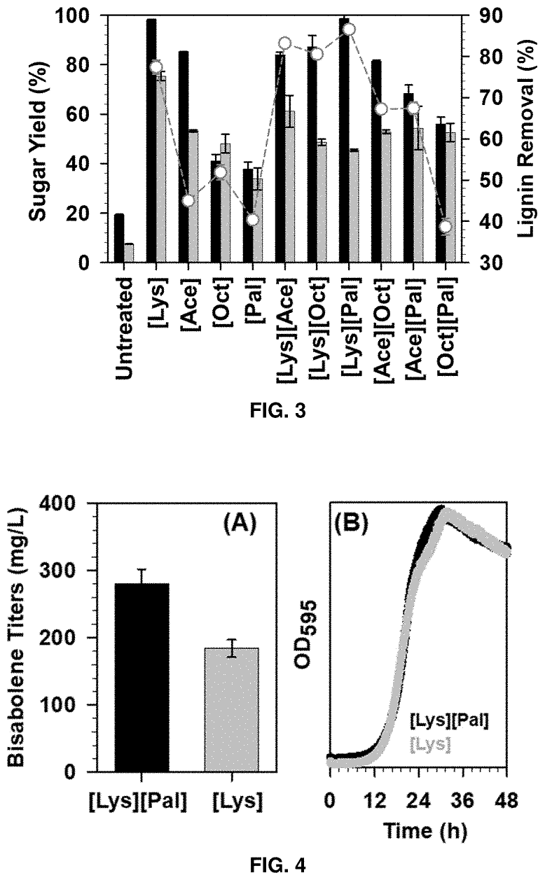

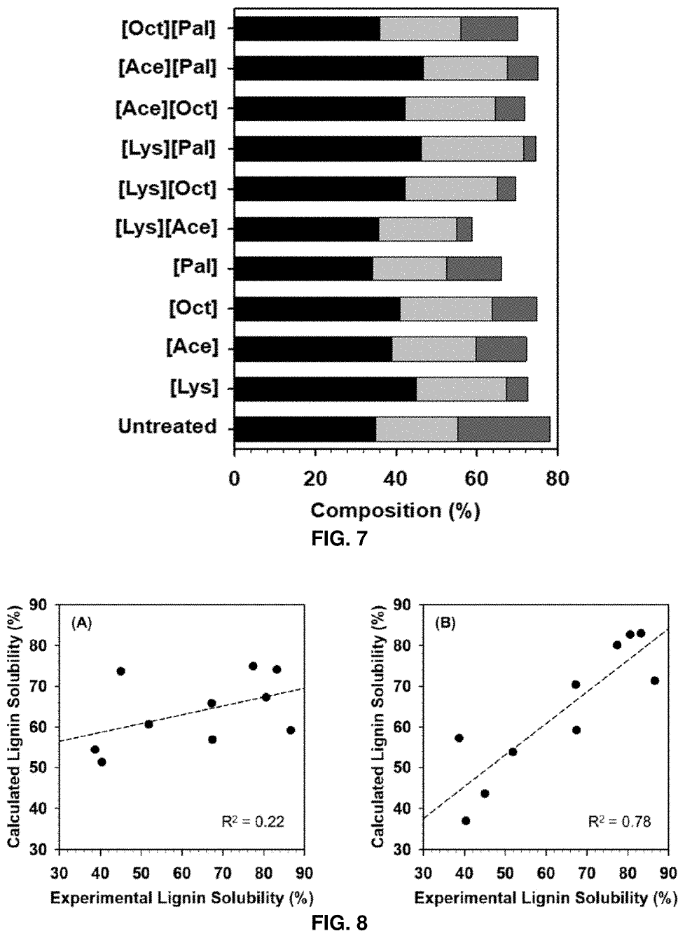

The change in holocellulose and lignin content was monitored before and after pretreatment to understand the effect of anions (FIG. 7). No glucan or xylan loss was observed for any of the IL/DSIL under study. Significant lignin loss (>65%) was recorded for most of the DSIL system with a maximum of 86.6% for [Ch][Lys][Pal] (FIG. 3). Discrepancies were observed in the predicted and experimental values in terms of lignin dissolution. For instance, [Ch][Lys] and [Ch][Ace] were predicted to dissolve lignin similarly, however, [Ch][Lys] and [Ch][Ace] distinguished with 77.4% and 45% delignification, respectively, after pretreatment under identical conditions. The disagreement between the COSMO-RS predictions and experimental values could be accounted for by considering the viscosity of the employed ILs, a critical factor in biopolymer dissolution. An increase in viscosity has been infamously celebrated to restrict mass transfer, hindering the solute dissolution (23, 24). Similarly, higher viscosities of [Ch][Oct] and [Ch][Pal] can explain the lower lignin removal efficiencies observed. The introduction of a second anion to form a DSIL improved the lignin removal capability through synergy. A 77.4% delignification degree achieved by [Ch][Lys] was promoted to 83.2%, 80.6%, and 86.6% by [Ch][Lys][Ace], [Ch][Lys][Oct], and [Ch][Lys][Pal], respectively. Remarkably, up to 49% enhancement in delignification was achieved for acetate-based DSILs containing octanoate or palmitate as second anion when compared to single anion containing [Ch][Ace]. Overall, the delignification competency was observed in the following order: [Ch][Lys][Pal]>[Ch][Lys][Ace]>[Ch][Lys][Oct]>[Ch][Lys]>[Ch][Ace][Pal]>[Ch][Ace][Oct]>[Ch][Oct]>[Ch][Ace]>[Ch][Pal]>[Ch][Oct][Pal].

Enzymatic hydrolysis of untreated and pretreated sorghum was performed (as described earlier) to evaluate the pretreatment efficiency of the cholinium-based IL/DSIL (FIG. 3). Pretreatment with these systems accelerated the enzyme activity compared to untreated sorghum (19.2% glucose and 7.5% xylose). [Ch][Lys][Pal] afforded maximum glucose release among all DSILs. The glucose release from the pretreated sorghum was in the following order: [Ch][Lys][Pal] (98.8%)˜ [Ch][Lys] (98.2%)>[Ch][Lys][Oct] (87.2%)>[Ch][Ace](85.2%)>[Ch][Lys][Ace] (84.0%)>[Ch][Ace][Oct] (81.4%)>[Ch][Ace][Pal] (68.4%)>[Ch][Oct][Pal] (56.0%)>[Ch][Oct] (41.1%)>[Ch][Pal] (37.9%). The efficacy of sugar release was perceived as the function of lignin removal efficiency for most ILs/DSILs. Higher delignification correlated to the ease of cellulose digestibility in the case of lysinate or palmitate containing DSIL.

The mismatch of the polarity and hydrophilicity of lysinate and palmitate in [Ch][Lys][Pal] could be pivotal for the observed pretreatment efficacy as reported earlier for other systems (25); however, this is essentially speculative at this stage. Biomass pretreated with acetate containing systems, on the other hand, resulted in better cellulose digestibility, although the delignification ability was poor. This could be a result of the reduced cellulose crystallinity and increased surface area accessibility effects caused by acetate-based systems. [Ch][Ace][Oct] or [Ch][Ace][Pal], containing soft and hard anions in single composition, is especially interesting in this regard because it may potentially promote two distinct mechanisms of pretreatment. This paves the path for further studies on biomass pretreatment using DSILs with varied molar ratios or multiple ions to meet the desired properties and go beyond the shortcomings of IL.

A one-pot biomass conversion technology comprising pretreatment, enzymatic hydrolysis, and fermentation was recently introduced by our research group (26, 27). This process eliminates the need for a water-wash step after pretreatment providing noteworthy economic and environmental advantages. The one-pot process, however, requires a biocompatible ionic liquid such as [Ch][Lys] to enable facile downstream processing. We investigated the viability of using [Ch][Lys][Pal] in a one-pot process. To do so, 20 wt % sorghum was mixed with 10 wt % [Ch][Lys][Pal] and 70 wt % DI water and heated at 140° C. for 3 h. The pH of the slurry was 8.4 after the pretreatment and it was adjusted to 5 using concentrated HCl before performing enzymatic saccharification as previously described. 63.9% glucose and 42.3% xylose yields were obtained in a one-pot configuration with [Ch][Lys][Pal]. The lower yields here could be attributed to diluted IL pretreatment (sorghum to IL 2:1 w/w) compared to preliminary screening (FIG. 3, sorghum to IL 1:4 w/w).

To evaluate the potential for microbial conversion of the sugars in this hydrolysate, an engineered strain of the yeast Rhodosporidium toruloides able to produce the biofuel precursor bisabolene was cultivated in the hydrolysates. The results indicate that the [Ch][Lys][Pal]hydrolysate obtained with a one-pot process can be directly used as cultivation media for R. toruloides, and the growth rates, bisabolene titers and substrate utilization yields are comparable to those obtained when the strain was cultivated in tryptic broth (FIG. 4).

In summary, we have developed unique IL systems comprising of ions with distinctive pretreatment mechanisms to improve the pretreatment efficacy. The existing global knowledge on pretreatment will provide insights on how to develop and control the physicochemical properties of eccentric combinations of mechanistically different ions in an IL that are also compatible with the downstream processes. We would like to emphasize that this work demonstrates a mere example of the humongous possibilities that one can design and apply not only for fractionation of biomass but also their further processing contributing to overall lower environmental and economic impact.

### Materials

All materials were used as supplied unless otherwise noted. Water was deionized, with specific resistivity of 18 MΩ·cm at 25° C., from Purelab Flex (ELGA, Woodridge, Ill.). Choline hydroxide (45% in methanol), acetic acid (>99.7%), octanoic acid (≥99%), sodium hydroxide pellets (≥97%), methanol, acetyl bromide (>99%), ammonium sulfate, dodecane, pentadecane, hydroxylamine hydrochloride (99%), sodium azide, 1-ethyl-3-methylimidazolium acetate, sulfuric acid (98%), and deuterated dimethyl sulfoxide were obtained from Sigma-Aldrich (St. Louis, Mo.). Ethanol (200 proof) was purchased from Decon Labs, Inc. (King of Prussia, Pa.). Sulfuric acid (72%) was procured from RICCA chemical company (Arlington, Tex.). Amresco, Inc. (Solon, Ohio) was the source of L-lysine monohydrate. Alkaline lignin and bisabolene were purchased from TCI (Portland, Oreg.). J. T. Baker, Inc. (Phillipsburg, N.J.) supplied hydrochloric acid and sodium citrate dihydrate, while citric acid monohydrate (≥99.99%) was obtained from Merck (Kenilworth, N.J.). Palmitic acid was supplied by Acros Organics (Fairlawn, N.J.).

Analytical standard grade glucose and xylose were also obtained from Sigma-Aldrich (St. Louis, Mo.) and used for calibration.

Biomass studied here were pine (Pinus radiata) and sorghum (Sorghum bicolor) (the sorghum was donated by Idaho National Labs (Idaho Falls, Id.). The biomass was dried for 24 h in a 40° C. oven. Subsequently, it was knife-milled with a 2 mm screen (Thomas-Wiley Model 4, Swedesboro, N.J.). The resulting biomass was then placed in a leak-proof bag and stored in a dry cool place.

Commercial cellulase (Cellic© CTec3) and hemicellulase (Cellic© HTec3) mixtures were provided by Novozymes, North America (Franklinton, N.C.).

### Syntheses of Ionic Liquids (ILs) and Double Salt ILs (DSILs)

All ILs and DSILs were synthesized by an acid-base reaction of cholinium hydroxide and corresponding acid or mixture of acids.

General synthesis of ILs. In an oven-dried round-bottomed flask (RBF) containing a Teflon-coated magnetic stirring bar, acid (0.05 mol) was weighed. The flask was mounted on an ice-bath and an additional funnel (sealed with septa) was attached to the RBF. N₂ was purged into the flask through additional funnel and allowed to flow for a while. Anhydrous methanol (100 mL) was added to the flask and stirred to dissolve the acid component. Following the dissolution, 0.05 mol cholinium hydroxide in methanol was transferred to the addition funnel and added dropwise to the stirring cold methanolic solution of acid. The mixture was then stirred for an additional 1 h. Majority of the methanol was removed under reduced pressure at 50-60° C. using a rotary evaporator. Remaining solvent (methanol and water, by product of acid base reaction) was further removed by freeze-drying the reaction mixture to obtain the desired ILs. The purity and identity of the ILs were determined and established by NMR.

General synthesis of DSILs. In an oven-dried round-bottomed flask (RBF) containing a Teflon-coated magnetic stirring bar, an equimolar mixture of acids (0.025 mol each) was weighed. The flask was mounted on an ice-bath and an additional funnel (sealed with septa) was attached to the RBF. N₂ was purged into the flask through additional funnel and allowed to flow for a while. Anhydrous methanol (100 mL) was added to the flask and stirred to dissolve the acid component. Following the dissolution, 0.05 mol cholinium hydroxide in methanol was transferred to the addition funnel and added dropwise to the stirring cold methanolic solution of acid. The mixture was then stirred for an additional 1 h. Majority of the methanol was removed under reduced pressure at 50-60° C. using a rotary evaporator. Remaining solvent (methanol and water, by product of acid base reaction) was further removed by freeze-drying the reaction mixture to obtain the desired DSILs. The purity and identity of the DSILs were determined and established by NMR.

### Biomass Pretreatment (Washing Method)

All pretreatment reactions were conducted in duplicate. 2 mm pine or sorghum samples and IL/DSIL were mixed in a 1:4 ratio (w/w) to afford a biomass loading of 20 wt % in a 15 mL capped glass pressure tube and pretreated for 3 h in an oil bath heated at 140° C. After pretreatment, samples were removed from the oil bath and allowed to cool. 10 mL DI water-ethanol (1:1 v/v) was slowly added to the biomass-IL slurry and mixed well. The mixture was transferred to 50 mL Falcon tubes and centrifuged at high speed (4000 rpm) to separate solids and remove any residual IL. The ethanol-water washed solid was freeze-dried to obtain dried pretreated biomass for further analysis.

### Enzymatic Saccharification

All enzymatic saccharifications were conducted in duplicate. Enzymatic saccharification of pretreated and untreated biomass was carried out using commercially available enzymes, Cellic® Ctec3 and Htec3 (9:1 v/v) from Novozymes, at 50° C. in a rotary incubator (Enviro-Genie, Scientific Industries, Inc.). All reactions were performed at 5 wt % biomass loading in a 15 mL centrifuge tube. The pH of the mixture was adjusted to 5 with 50 mM sodium citrate buffer supplemented with 0.02% sodium azide to prevent microbial contamination. The total reaction volume included a total protein content of 10 mg per g biomass. The amount of sugars released was measured by HPLC as described previously.

### Compositional Analysis-Glucan and Xylan

All compositional analysis experiments were conducted in duplicate. Compositional analysis of biomass before and after pretreatment was performed using NREL two-step acid hydrolysis protocols (LAP) LAP-002 and LAP-005 (A. Sluiter, National Renewable Energy Laboratory (NREL) Analytical Procedures, 2004). Briefly, 200 mg of biomass and 2 mL of 72% sulfuric acid (H₂SO₄) were incubated at 30° C. while shaking at 200 rpm for 1 h. The solution was diluted to 4% H₂SO₄ with 56 mL of DI water and autoclaved at 121° C. for 1 h. The reaction was quenched by cooling down the flasks before removing the solids by filtration. Glucose and xylose concentrations were determined from the filtrate using HPLC (as described previously). The amount of glucan and xylan was calculated from the glucose and xylose content multiplied by the anhydro correction factors of 162/180 and 132/150, respectively.

### Compositional Analysis-Lignin

All compositional analysis experiments were conducted in duplicate. Acetyl bromide-based lignin assay method was employed to determine the lignin content in IL/DSIL pretreated sorghum samples as reported previously (R. S. Fukushima, M. S. Kerley, M. H. Ramos, J. H. Porter, R. L. Kallenbach, Anim. Feed Sci. Technol. 2015, 201, 25). 10 mg alcohol insoluble biomass residues were weighed in a 2 mL screw cap tubes vial. 1 mL 25% (v/v) acetyl bromide in glacial acetic acid was added to the vials containing biomass samples (Caution: must be operated in fume hood). The vials were sealed and incubated at 50° C. for 2 h with a rotational motion. After 2 h of incubation, vials were cooled in an ice bath for about 5 minutes before centrifuging the samples at 14,000 rpm for 5 minutes. The UV absorbance (at 280 nm) was measured by diluting 6 μL of supernatant with 60 μL master solution (obtained by mixing 48 μL acetic acid, 9.5 μL 2M NaOH and 1.7 μL 0.5M hydroxylamine hydrochloride) and 200 μL glacial acetic acid. It is important to always draw liquids (pipetting) at least 3 times to assure same amounts of liquid transfer.

The lignin concentration was measured by calibration curve method. In a 2 mL screw cap tubes vial, 10 mg alkaline lignin was treated with 1 mL 25% (v/v) acetyl bromide in glacial acetic acid and incubated at 50° C. for 2 h with a rotational motion. After 2 h of incubation, vials were cooled in an ice bath for about 5 minutes before centrifuging the samples at 14,000 rpm for 5 minutes. Standard samples were prepared by diluting 1, 2, 4, and 6 μL of supernatant with 60 μL master solution and 200 μL glacial acetic acid. UV absorbance was measured at 280 nm and compared against blank (60 μL master solution and 200 μL glacial acetic acid).

### One-Pot Biomass Pretreatment and Saccharification

All pretreatment reactions were conducted in duplicate. 2 mm sorghum samples, cholinium lysinate palmitate ([Ch][Lys][Pal]) DSIL, and water were mixed in a 2:1:7 ratio (w/w) (20 wt % biomass loading) in a 15 mL capped glass pressure tube and pretreated for 3 h in an oil bath heated at 140° C. The prior mixing ensures homogeneous mixtures of biomass and IL. After pretreatment, samples were removed from the oil bath and cooled to room temperature. The pH of the cold pretreated mixture was noted and adjusted to 5 with hydrochloric acid (HCl). Enzymatic saccharification was run at 50° C. for 72 hours on an Enviro Genie SI-1200 rotator platform (Scientific Industries, Inc., Bohemia, N.Y.). The enzyme mixtures Cellic® CTec3 and HTec3 (9:1 v/v) were used at a loading of 10 mg protein/g biomass. The pretreatment efficiency in terms of sugar release was analyzed on an Agilent HPLC 1260 infinity system (Santa Clara, Calif., United States) equipped with a Bio-Rad Aminex HPX-87H column and a Refractive Index detector. An aqueous solution of sulfuric acid (4 mM) was used as the eluent (0.6 mL min⁻¹, column temperature 60° C.).

### Microbial Cultivations in Biomass Hydrolysates

An engineered strain of the oleaginous yeast Rhodosporidium toruloides that produces the jet fuel precursor bisabolene, named GB2.0, was used to test the biocompatibility of the generated hydrolysates after pretreatment and saccharification. This strain is deposited in the Joint BioEnergy Institute public registry (website for: public-registry.jbei.org) with the identification number JBx_086452. The hydrolysates were supplemented with ammonium sulfate (NH₄SO₄; from a 100x stock solution for a final concentration of 5 g/L) and filtered through 0.45 μm surfactant-free cellulose acetate membranes after adjusting the pH to 7 with concentrated NaOH. Hydrolysates diluted by 50% with water containing the same amount of NH₄SO₄ were also generated.

R. toruloides was first cultivated in tubes containing 10 mL of tryptic soy broth from freshly streaked plates and incubated at 30° C. and 200 rpm for 24 hours. To start the experiment, 5 μL of the seed cultures were combined with 145 μL of the filtered hydrolysates or fresh tryptic soy broth as a control in a lidded 96-well plate and incubated at 30° C. with shaking using a DTX880 multiplate reader (Beckton-Coulter, USA). The optical density at 595 nm was measured each 5 minutes for 48 hours and used to obtain the average maximum cell biomass (the highest OD 595 nm value) and the average growth rate (the slope of growth curves during the exponential phase, after plotting the natural logarithm of OD values versus time) from each condition. The cultivations were performed by triplicate.

For the bisabolene production experiments, 780 μL of the pH-adjusted and filtered hydrolysates were transferred to 48-well FlowerPlates (m2p labs, Germany) containing 20 μL of cells and 200 μL of a dodecane overlay, and covered with sterile AeraSeal films (Excel Scientific, USA). The plates were incubated for 7 days in a humidity-controlled incubator with orbital shaking at 900 rpm. The entire contents of each well were collected in 1.5 mL tubes and the dodecane layer, supernatant, and cells were separated by centrifugation and each fraction was kept at −20° C. until analysis. The cell pellets were then resuspended in 800 μL of water, diluted forty-fold with water, and 100 μL were transferred to 96-well plates to measure final optical density at 600 nm using a SpectraMax Plus 384 reader (Molecular Devices, USA). All cultivations were performed in triplicate.

To quantify bisabolene produced in the flowerplate cultivations, the dodecane overlays obtained at the end of the experiments were diluted in pure dodecane spiked with 40 mg/L of pentadecane, used as an internal standard. The samples were then analyzed by GC-MS using an Agilent Technologies 6890N system equipped with a 5973-mass selective detector and a DB-5 ms column (30 m×250 μm×0.25 μm, Agilent Technologies, USA). 1 μl injections with a splitless setting were used on a GC oven program consisting of 100° C. for 0.75 min, followed by a ramp of 20° C. per min until 300° C., and held 1 min at 300° C. Injector and MS quadrupole detector temperatures were 250° C. and 150° C., respectively. The bisabolene concentrations reported here correspond to the actual concentrations in the dodecane layer, calculated by integration of the peak area values obtained in selective ion monitoring mode and compared to the areas obtained from a calibration curve made with pure bisabolene.

### COSMO-RS Details

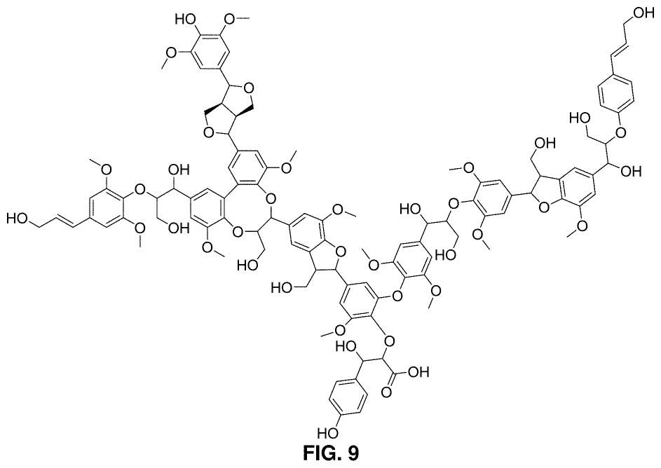

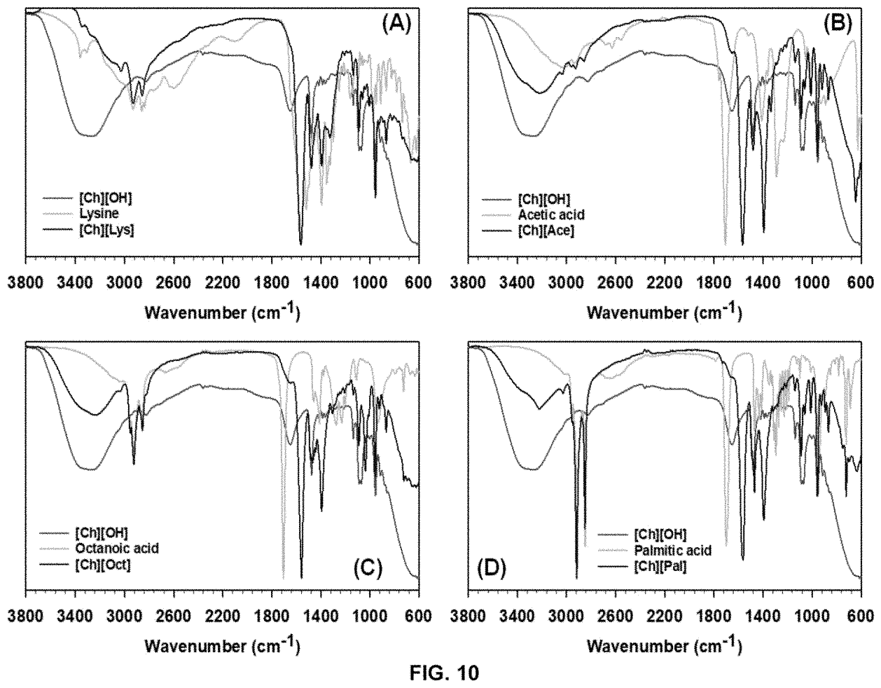

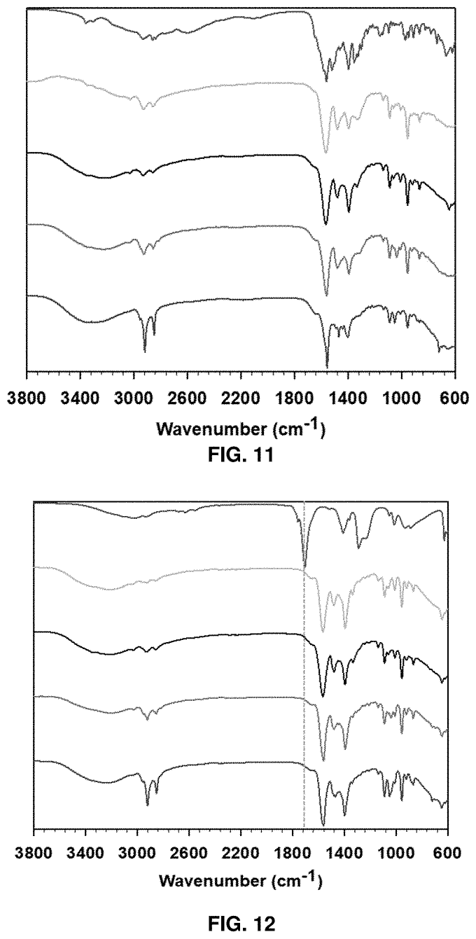

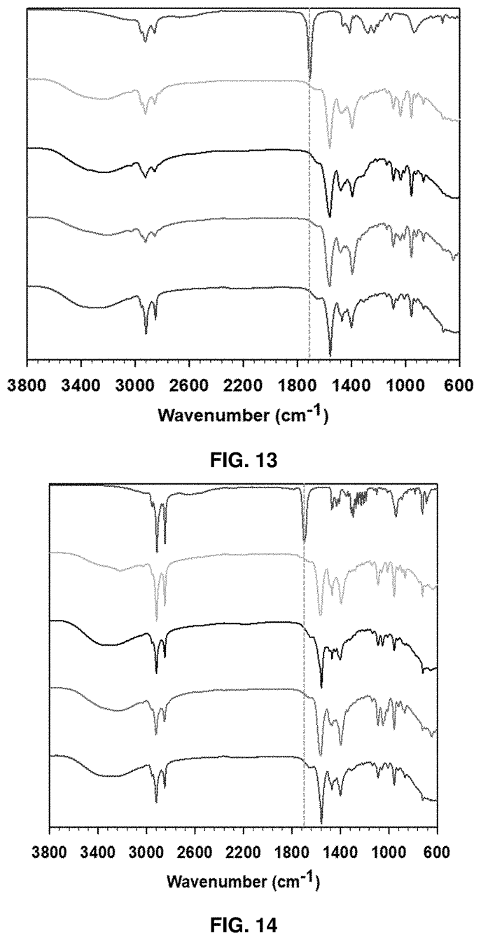

Using the COSMO-RS calculations, the dissolution and/or interaction of lignin in the cholinium-based ILs and DSILs was predicted. To perform these calculations, the initial structures of lignin (FIG. 9), ILs, and DSILs were drawn in the Avogadro freeware software (M. D. Hanwell, D. E. Curtis, D. C. Lonie, T. Vandermeersch, E. Zurek, G. R. Hutchison, J. Cheminformatics 2012, 4, 17). The structures of all the investigated molecules were optimized using Gaussian09 package at B3LYP (Becke 3-parameter hybrid functional combined with the Lee-Yang-Parr correlation) theory and 6-311+G(d,p) basis set (Gaussian 09, Revision D.01, M. J. Frisch, et al., Gaussian, Inc., Wallingford Conn., 2009; Y. Zhang, H. He, K. Dong, M. Fan, S. Zhang, RSC Adv. 2017, 7, 12670). To confirm the energy minima of the optimized structure and verify the presence of any imaginary frequency, frequency calculations have been performed at the same level of theory and no imaginary frequencies were present after optimization.

After geometry optimization step, further, the COSMO file was generated using the BVP86/TZVP/DGA1 level of theory (M. Gonzalez-Miquel, M. Massel, A. DeSilva, J. Palomar, F. Rodriguez, J. F. Brennecke, J. Phys. Chem. B 2014, 118, 11512). The ideal screening charges on the molecular surface were computed using the same level of theory i.e., BVP86 through the “scrf=COSMORS” keyword (M. Mohan, V. V. Goud, T. Banerjee, Fluid Phase Equilibr. 2015, 395, 33). The generated COSMO files were then used as an input in the COSMOtherm (version 19.0.1, COSMOlogic, Leverkusen, Germany) package with BP_TZVP_19 parametrization (F. Eckert, A. Klamt, AIChE J. 2002, 48, 369). In COSMO-RS calculations, the molar fraction of lignin was set as 0.2, whereas the molar fraction of solvents was set to 0.8 to mimic the experimental pretreatment setup with a biomass to IL loading ratio of 1:4 (w/w). The activity coefficient of component i is associated with the chemical potential Pi and expressed as (K. A. Kurnia, S. P. Pinho, J. A. P. Coutinho, Ind. Eng. Chem. Res. 2014, 53, 12466),

In

$$( {γ}_{i} ) = ( \frac{{μ}_{i} - {μ}_{i}^{0}}{RT} )$$

where, μi⁰ is the chemical potential of the pure component i, R is the real gas constant and T is the absolute temperature. The details of COSMO-RS calculation are provided in the COSMOtherm's user manual (F. Eckert, A. Klamt, COSMOtherm, version C3.0 release 19.0.1. COSMOlogic GmbH & Co KG: Leverkusen, Germany, 2019).

### Thermal Gravimetric Analysis

Thermal behavior was determined using a Mettler Toledo Stare TGA/DSC1 unit (Mettler Toledo, Leicester, UK) under nitrogen. Samples between 3 and 10 mg were placed in alumina crucibles (70 μL) and heated from room temperature to 75° C. at a heating rate of 10° C./min. An isotherm at 75° C. was maintained for 30 min to eliminate all volatiles, if any. After the isothermal step, the temperature was ramped to 800° C. at a heating rate of 10° C./min. The data was analyzed using STARe Evaluation software.

TABLE 1
Thermal gravimetric analysis of the ILs and DSILs.

| IL/DSIL | T_{5%} (° C.) | T_{50%} (° C.) |
|---|---|---|
| [Ch][Lys] | 155.9 | 218.1 |
| [Ch][Ace] | 173.1 | 211.4 |
| [Ch][Oct] | 180.7 | 209.5 |
| [Ch][Pal] | 177.7 | 225.3 |
| [Ch][Lys][Ace] | 159.9 | 210.2 |
| [Ch][Lys][Oct] | 150.3 | 211.4 |
| [Ch][Lys][Pal] | 147.0 | 228.5 |
| [Ch][Ace][Oct] | 170.1 | 211.4 |
| [Ch][Ace][Pal] | 181.1 | 212.3 |
| [Ch][Oct][Pal] | 176.8 | 209.6 |

T_{5%}: Decomposition temperature of 5% sample.
T_{50%}: Decomposition temperature of 50% sample.

¹H NMR and ¹³C NMR are performed for cholinium lysinate, cholinium acetate, cholinium octanoate, cholinium palmitate, cholinium lysinate acetate, cholinium lysinate octanoate, cholinium lysinate palmitate, cholinium acetate octanoate, cholinium acetate palmitate, cholinium octanoate palmitate. The results are shown in U.S. Provisional Patent Application Ser. No. 63/129,494, filed Dec. 22, 2020.

### Example 2

Pretreatment of Biomass with IL/DSIL Mixtures at High Biomass Loading of 20 wt %

The biomass was pretreated with IL/DSIL mixtures at high biomass loading of 20 wt %. After the pretreatment, biomass was washed thoroughly with water and freeze dried. The freeze-dried material was further subjected to EH with cellulase and hemicellulase cocktails. Solids were separated from the hydrolysate, washed with water, and freeze-dried to obtain various lignin fractions.

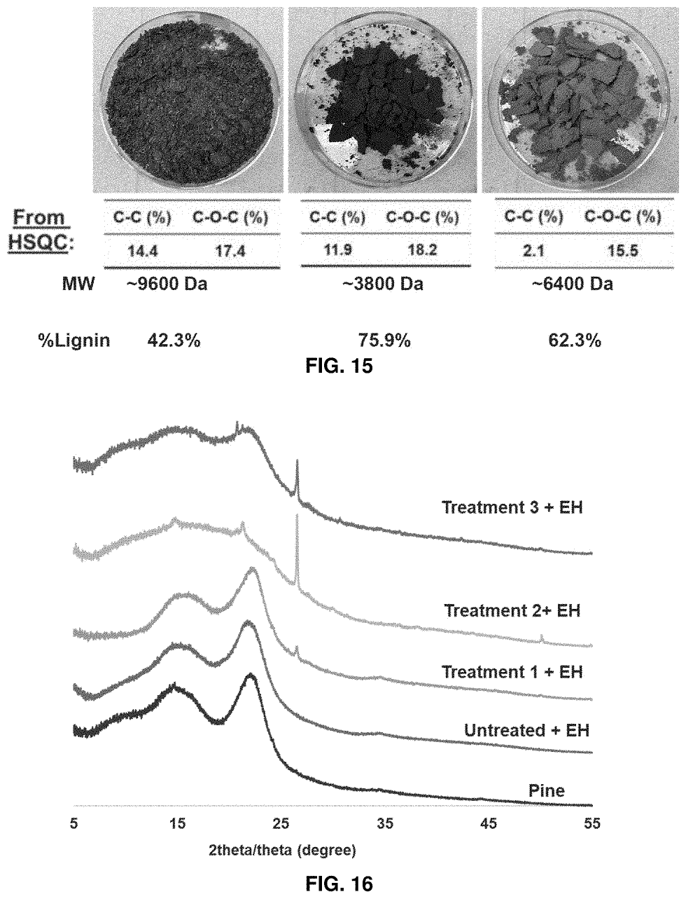

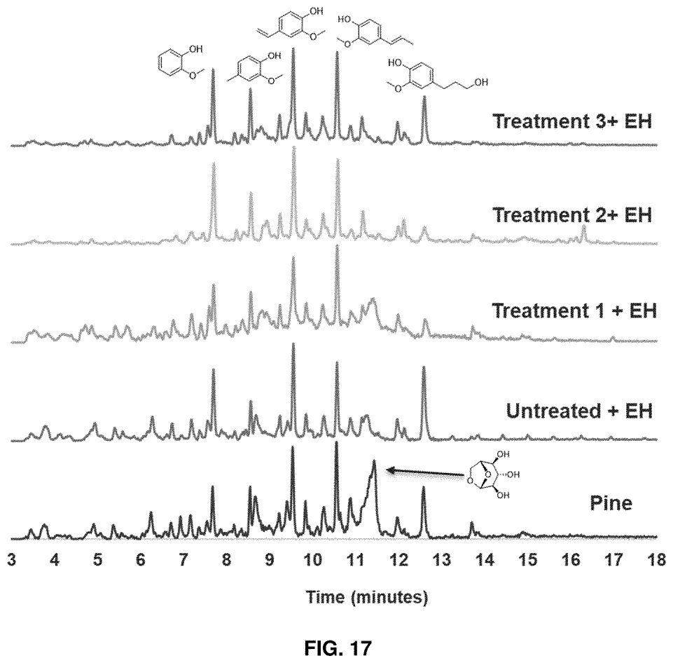

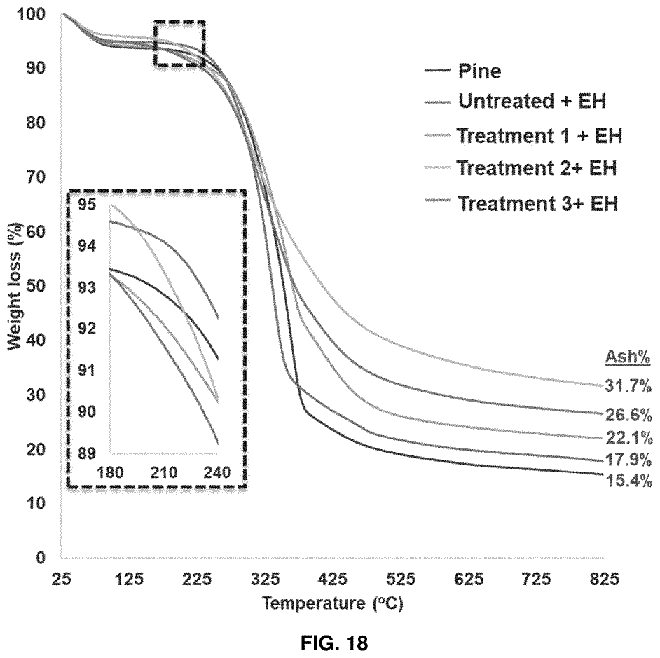

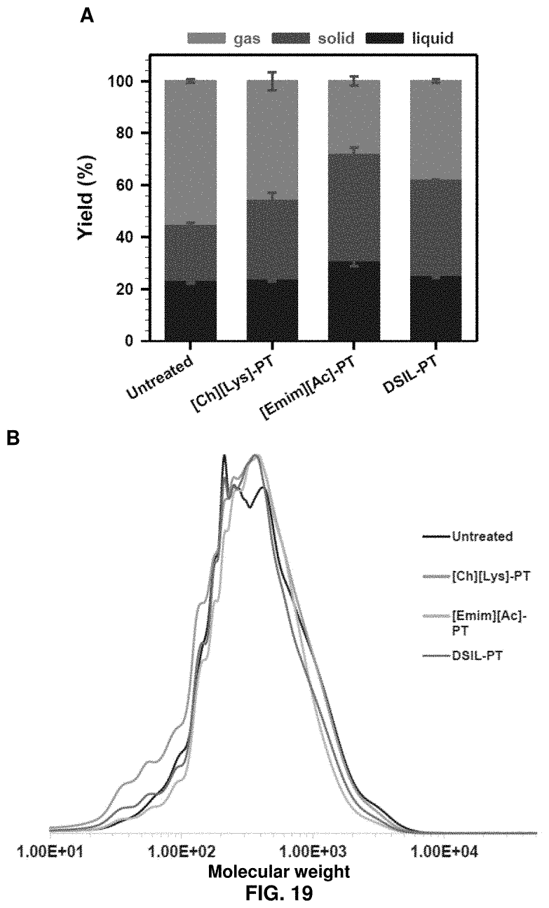

FIG. 15 shows various lignin obtained from Pine biomass after IL/DSIL pretreatment and enzymatic hydrolysis (EH). FIG. 16 shows powder X-ray diffraction patterns of lignin obtained after various IL/DSIL treatment demonstrating decrease in the cellulosic content and change in crystalline phases. EH is enzymatic hydrolysis. FIG. 17 shows pyro-GC analysis of the lignin obtained after various treatments showing the retention of units and linkages after treatment 3 as in the native pine. The sugar component in the pine is reduced after enzymatic hydrolysis (EH). FIG. 18 shows thermogravimetric analysis of lignin obtained after various treatments demonstrating distinct thermal profile. FIG. 19 shows the Pd/ZrP catalysis on pine. (A) The results from different pretreatment conditions. (B) The distribution of molecules by molecular weight after pretreatment. The reaction conditions used are: IL-processed-pine lignin (0.35 g), Pd/ZrP (0.1 g), iPrOH:MeOH (2:1 v/v, 10 mL), 300° C., N₂ (18 bar), 500 rpm. Treatments 1, 2, and 3, as indicated in the figures, are (1) pretreatment with cholinium lysinate, (2) pretreatment with Emim acetate, and (3) pretreatment with DSIL (mixture of Emim acetate and cholinium lysinate), respectively.

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to deconstruct a biomass: the method comprising: (a) introducing a solvent comprising a plurality of salt ionic liquid (PSIL) to a biomass to dissolve at least part of solid biomass in the solvent; wherein the PSIL is an organic salt comprising three or more ions, and the PSIL comprises: (i) a hard anion ionic liquid (IL) and a soft anion IL, (ii) at least one IL having a pKa value of equal to or higher than 10, or (iii) at least one IL has a low hydrogen bond donor ability; (b) optionally introducing an enzyme and/or a microbe to the solubilized biomass mixture such that the enzyme and/or microbe produces a sugar from the solubilized biomass mixture; and, (c) optionally separating the sugar from the solubilized biomass mixture.

2. The method of claim 1, wherein the PSIL is a double salt ionic liquid (DSIL).

3. The method of claim 1, wherein the PSIL comprises an ionic liquid (IL) having the formula [aC₁+bC₂+ . . . +zCₙ][αA₁+βA₂+ . . . +ωAₙ₋₁+(1−α−β− . . . −ω)Aₙ]; wherein C₁, C₂, . . . and Cₙ are organic cations and at least one organic cation is an alkylammonium, an arylammonium, an allylammonium, an imidazolium, a pyridinium, a phosphonium, a sulphonium, or a combination thereof; A₁, A₂, . . . Aₙ are anions, wherein at least one of the anions is a hard anion comprising a carboxylic acid or an amino acid; a, b, . . . and z are independently a number from about 0 to 20; and a sum of a, b, . . . and z is greater than 0; a sum of α+β+ . . . +ω is a number greater than zero.

4. The method of claim 3, wherein the PSIL comprises an ionic liquid (IL) having the formula [mC₁+nC₂][xA₁+(1−x)A₂)]; wherein C₁ and C₂ are organic cations and at least one organic cation is an alkylammonium, an arylammonium, an allylammonium, an imidazolium, a pyridinium, a phosphonium, a sulphonium, or a combination thereof; A₁ and A₂ are anions, wherein at least one of the anions is a hard anion comprising a carboxylic acid or an amino acid; m and n are independently a number from about 0 to 20; and a sum of m and n is greater than 0; x is a number from about 0.01 to 0.99.

5. The method of claim 3, wherein the carboxylic acid is an acetate or propionate.

6. The method of claim 3, wherein the amino acid is a lysine or glycine.

7. The method of claim 1, wherein the IL is a liquid at a temperature from about −80° C. to about 150° C.

8. The method of claim 1, wherein the solvent has a viscosity having a value equal to or less than about 50 cP at a temperature of about 90° C.

9. The method of claim 1, wherein the solvent has a viscosity having a value equal to or less than about 600 cP at a temperature of about 25° C.

10. The method of claim 1, wherein the combination of hard anion renders the hydrogen bond basicity of the IL at equal to or more than about 0.25 at about 90° C.

11. The method of claim 1, wherein the solubilized biomass mixture produces fewer guaiacol and/or derivatives thereof, compared to a pretreatment using only one salt ionic liquid.

12. The method of claim 1, wherein the solubilized biomass mixture has fewer guaiacol and/or derivatives thereof, compared to a pretreatment using only one salt ionic liquid.

13. The method of claim 1, wherein the method does not comprise, or lacks, introducing or adding a capping agent to the biomass, solvent, and/or solubilized biomass mixture.

14. The method of claim 1, wherein the method produces a profile of lignin that is substantially similar to the profile of native or native-like lignin.

15. The method of claim 1, wherein the method produces a peak similar to that for untreated or pine biomass for the molecule:

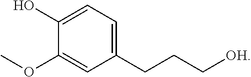

16. The method of claim 1, wherein equal to or more than 50% of the lignin in the solubilized biomass mixture has a molecular weight within a range of 1000 to 15000 Da.
